(19) United States
(12) Patent Application Publication
(10) Pub. No.: US 2019/0328633 A1
(43) Pub. Date: Oct. 31, 2019

(54) COMPOSITIONS AND METHODS FOR CLEANSING KERATIN FIBERS

(71) Applicant: L'OREAL, Paris (FR)

(72) Inventors: Halil GEVGILILI, Weehawken, NJ (US); Jun LIANG, Staten Island, NY (US); Heather LEE, Wayne, NJ (US); Semra SENTURK-OZER, Westfield, NJ (US)

(21) Appl. No.: 16/399,598

(22) Filed: Apr. 30, 2019

Related U.S. Application Data
(60) Provisional application No. 62/664,652, filed on Apr. 30, 2018.

(51) Int. Cl. A61K 8/19; A61K 8/365; A61K 8/81; A61Q 5/02; A61Q 5/12

(52) U.S. Cl. CPC A61K 8/19; A61K 8/365; A61K 8/8158; A61Q 5/02; A61K 8/416; A61K 2800/222; A61K 2800/882; A61K 2800/87; A61Q 5/12

(57) ABSTRACT

The disclosure relates to foaming compositions for cleansing keratin fibers, in particular human keratin fibers such as the hair, as well as methods for cleansing keratin fibers using the foaming compositions according to the disclosure.

### TECHNICAL FIELD

The disclosure relates to foaming compositions for cleansing keratin fibers, in particular human keratin fibers such as the hair, as well as methods for cleansing keratin fibers using the foaming compositions according to the disclosure.

### BACKGROUND

Conventional compositions for cleansing keratin fibers, such as shampoos, are widely known and used. Such compositions are typically in the form of a liquid, which the user works into a foam by lathering on the hair and scalp. However, consumers desire improved consistency and sensory properties, such as foaming, in the application stage.

Carbon dioxide-generating compositions, such as sodium bicarbonate, can be used to provide an effervescent effect in personal care compositions such as shampoo. However, the use of such carbon dioxide-generating compounds in personal care compositions, and in particular liquid shampoo compositions, presents many challenges. For example, such compounds are not stable in aqueous systems due to premature decomposition to form carbon dioxide gas, which particularly readily decompose when exposed to an acid.

To prevent premature decomposition, the carbon dioxide-generating compounds can be isolated from an acidic medium into a separate phase, e.g., a booster composition. However, while it is desirable to have a higher concentration of such compounds in a booster composition, they are generally soluble in water only at a concentration of up to around 8-9%. Moreover, such solutions have a consistency that is too thin for handling and use in a cleansing composition.

Therefore, it is an object of the disclosure to provide foaming compositions with carbon dioxide-generating compounds for cleansing keratin fibers, which have acceptable properties of stability, consistency, and sensoriality. The foaming compositions may be particularly advantageous for users who have oily hair, mineral buildup, or as mid-cycle treatment for unwashed hair.

### SUMMARY

The present disclosure relates to systems, compositions, and methods for cleansing keratin fibers, such as the hair. The systems, compositions, and methods combine at least one carbon dioxide-generating compound with at least one anionic terpolymer in a booster composition which can be used in conjunction with shampoo compositions to provide effervescent cleansing compositions. The present disclosure also relates to kits and containers for such systems and compositions.

Thus, according to various embodiments, the disclosure relates to systems comprising: (a) a booster composition comprising (i) at least one carbon dioxide-generating compound, and (ii) at least one anionic terpolymer; and (b) a shampoo composition; wherein the booster composition and the shampoo composition are combined to form an effervescent cleansing composition at or near the time of use. According to various embodiments of systems, the at least one carbon dioxide-generating compound is chosen from carbonate or bicarbonate salts of alkaline metals or alkaline earth metals, and the at least one anionic terpolymer is chosen from linear or branched and/or crosslinked terpolymers comprising at least one monomer (1) bearing an acid function in free form, partially or totally salified with a nonionic monomer (2) chosen from N,N-dimethylacrylamide and 2-hydroxyethyl acrylate, and at least one polyoxyethylenated alkyl acrylate monomer (3) of formula (I), as defined herein; and the shampoo composition comprises at least one cleansing surfactant and at least one acid. In yet further embodiments of systems, the at least one carbon dioxide-generating compound comprises sodium bicarbonate, and the at least one anionic terpolymer comprises polyacrylate crosspolymer-6.

According to further embodiments, the disclosure relates to methods of cleansing keratin fibers with the systems described, the methods comprising mixing: (a) a booster composition comprising (i) at least one carbon dioxide-generating compound, and (ii) at least one anionic terpolymer; and (b) a shampoo composition to form an effervescent cleansing composition, applying the effervescent cleansing composition to the keratin fibers, and optionally rinsing the keratin fibers. According to various embodiments of methods of cleansing, the at least one carbon dioxide-generating compound is chosen from carbonate or bicarbonate salts of alkaline metals or alkaline earth metals, and the at least one anionic terpolymer is chosen from linear or branched and/or crosslinked terpolymers comprising at least one monomer (1) bearing an acid function in free form, partially or totally salified with a nonionic monomer (2) chosen from N,N-dimethylacrylamide and 2-hydroxyethyl acrylate, and at least one polyoxyethylenated alkyl acrylate monomer (3) of formula (I), as defined herein; and the shampoo composition comprises at least one cleansing surfactant and at least one acid. In yet further embodiments of methods of cleansing, the at least one carbon dioxide-generating compound comprises sodium bicarbonate, and the at least one anionic terpolymer comprises polyacrylate crosspolymer-6.

According to yet further embodiments, the disclosure relates to kits, the kits comprising (1) a first compartment comprising a booster composition comprising at least one carbon dioxide-generating compound and at least one anionic terpolymer; and (2) a second compartment comprising a shampoo composition; and wherein the booster composition and the shampoo composition are in mutually separate compartments. According to various embodiments of kits, the at least one carbon dioxide-generating compound is chosen from carbonate or bicarbonate salts of alkaline metals or alkaline earth metals, and the at least one anionic terpolymer is chosen from linear or branched and/or crosslinked terpolymers comprising at least one monomer (1) bearing an acid function in free form, partially or totally salified with a nonionic monomer (2) chosen from N,N-dimethylacrylamide and 2-hydroxyethyl acrylate, and at least one polyoxyethylenated alkyl acrylate monomer (3) of formula (I), as defined herein; and the shampoo composition comprises at least one cleansing surfactant and at least one acid. In yet further embodiments of kits, the at least one carbon dioxide-generating compound comprises sodium bicarbonate, and the at least one anionic terpolymer comprises polyacrylate crosspolymer-6.

According to still further embodiments, the disclosure relates to multi-chamber containers comprising first and second chambers that are separate from one another, wherein: (1) the first chamber comprises a booster composition comprising at least one carbon dioxide-generating compound and an anionic terpolymer; and (2) the second chamber comprises a shampoo composition; and wherein the container is configured so that the booster composition is dispensed from the first chamber and the shampoo composition is dispensed from the second chamber substantially simultaneously to form an effervescent cleansing composition. According to various embodiments of multi-chamber containers, the at least one carbon dioxide-generating compound is chosen from carbonate or bicarbonate salts of alkaline metals or alkaline earth metals, and the at least one anionic terpolymer is chosen from linear or branched and/or crosslinked terpolymers comprising at least one monomer (1) bearing an acid function in free form, partially or totally salified with a nonionic monomer (2) chosen from N,N-dimethylacrylamide and 2-hydroxyethyl acrylate, and at least one polyoxyethylenated alkyl acrylate monomer (3) of formula (I), as defined herein; and the shampoo composition comprises at least one cleansing surfactant and at least one acid. In yet further embodiments of multi-chamber containers, the at least one carbon dioxide-generating compound comprises sodium bicarbonate, and the at least one anionic terpolymer comprises polyacrylate crosspolymer-6.

In still further embodiments, the disclosure relates to containers comprising a booster composition for use in conjunction with a shampoo in order to provide an effervescent cleansing composition for keratin fibers, wherein the container comprises from about 5-25% sodium bicarbonate and from about 1-3% polyacrylate crosspolymer-6, by weight of the booster composition.

### DESCRIPTION

The present disclosure relates to systems for providing foaming compositions for cleansing keratin fibers, in particular human keratin fibers such as the hair, as well as methods for cleansing keratin fibers using the systems and foaming compositions according to the disclosure.

The systems according to the disclosure comprise a first booster composition and a second shampoo composition that are mixed at or near the time of use to provide a hair cleansing composition having a foaming effect. The disclosure also relates in various embodiments to a booster composition that can be used in conjunction with a shampoo composition. The disclosure also relates to a kit comprising a booster composition according to the disclosure and a liquid shampoo composition, as well as a multi-chamber container comprising a booster composition according to the disclosure in one chamber and a liquid shampoo composition in a separate chamber. The disclosure further relates to containers comprising a booster composition according to the disclosure.

### Systems and Methods

Systems according to the disclosure comprise a first booster composition and a second shampoo composition that are mixed at or near the time of use to provide an effervescent hair cleansing composition. Methods according to the disclosure comprise using the systems and compositions described for cleansing keratin fibers, such as hair.

### Booster Composition

The booster composition comprises at least one carbon dioxide-generating compound and at least one anionic terpolymer.

The carbon dioxide-generating compound may be chosen from, for example, at least one carbonate or bicarbonate salt of at least one alkaline metal or alkaline earth metal. Non-limiting examples of carbonates and bicarbonates that can be used include sodium carbonate, potassium carbonate, magnesium carbonate, calcium carbonate, sodium bicarbonate, potassium bicarbonate, magnesium bicarbonate, calcium bicarbonate, as well as mixtures thereof.

The carbon dioxide-generating compound may be present in the booster composition in an amount sufficient to provide a desired level of foaming when used in conjunction with a shampoo composition. By way of example, the carbon dioxide-generating compound may be present in the booster composition in an amount up to 50%, such as up to 45%, up to 40%, up to 35%, up to 30%, up to 29%, up to 28%, up to 27%, up to 26%, up to 25%, up to 24%, up to 23%, up to 22%, up to 21%, up to 20%, up to 19%, up to 18%, up to 17%, up to 16%, up to 15%, up to 14%, up to 13%, up to 12%, up to 11%, up to 10%, up to 9%, up to 8%, up to 7%, up to 6%, up to 5%, up to 4%, up to 3%, up to 2%, or up to 1%, by weight of the booster composition. It is to be understood that the carbon dioxide-generating compound can be present in the booster composition in a range having any of the aforementioned numbers as either a low or high end of the range.

For example, in one embodiment, the carbon dioxide-generating compound is present in an amount ranging from 1-30%, by weight of the booster composition. In further exemplary embodiments, the carbon dioxide-generating compound is present in an amount ranging from 5-25%, such as from 5-20%, from 10-25%, from 10-20%, from 15-25%, or from 15-20%, including ranges and sub-ranges therebetween, by weight of the booster composition. In other embodiments, the carbon dioxide-generating compound is present in an amount of at least 10%, such as at least 15%, at least 20%, at least 25%, or at least 30% by weight, based on the weight of the booster composition.

The anionic terpolymer may be a linear or branched and/or crosslinked terpolymer, of at least one monomer (1) bearing an acid function in free form, which is partially or totally salified with a nonionic monomer (2) chosen from N,N-dimethylacrylamide and 2-hydroxyethyl acrylate and at least one polyoxyethylenated alkyl acrylate monomer (3) of formula (I) below:

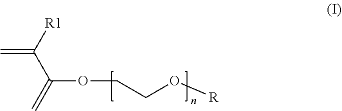

in which:

R1 represents a hydrogen atom,

R represents a linear or branched C2-C5 alkyl radical, and

n represents a number ranging from 1 to 10.

The term “branched polymer” denotes a non-linear polymer which bears pendent chains so as to obtain, when this polymer is dissolved in water, a high degree of entanglement leading to very high viscosities, at a low speed gradient.

The term “crosslinked polymer” denotes a non-linear polymer which is in the form of a three-dimensional network that is insoluble in water but swellable in water, leading to the production of a chemical gel.

The acid function of the monomer (1) may be a sulfonic acid or phosphonic acid function, said functions being in free or partially or totally salified form.

The monomer (1) may be chosen from styrenesulfonic acid, ethylsulfonic acid and 2-methyl-2-[(1-oxo-2-propenyl]amino]-1-propanesulfonic acid (also known as acryloyldimethyl taurate), in free or partially or totally salified form. It may be present in the anionic terpolymer in molar proportions ranging from 5 mol % to 95 mol %, such as from 10 mol % to 90 mol %. The monomer (1) may be 2-methyl-2-[(1-oxo-2-propenyl)amino]-1-propanesulfonic acid in free or partially or totally salified form.

The acid function in partially or totally salified form will preferably be an alkali metal salt such as a sodium or potassium salt, an ammonium salt, an amino alcohol salt such as a monoethanolamine salt, or an amino acid salt such as a lysine salt.

The monomer (2) may be present in the anionic terpolymer in molar proportions ranging from 4.9 mol % to 90 mol %, such as from 9.5 mol % to 85 mol %, or from 19.5 mol % to 75 mol %.

In formula (I), examples of linear C8-C16 alkyl radicals that may be mentioned include octyl, decyl, undecyl, tridecyl, tetradecyl, pentadecyl and hexadecyl.

In formula (I), examples of branched C8-C16 alkyl radicals that may be mentioned include 2-ethylhexyl, 2-propylheptyl, 2-butyloctyl, 2-pentylnonyl, 2-hexyldecyl, 4-methylpentyl, 5-methylhexyl, 6-methylheptyl, 15-methylpentadecyl, 16-methylheptadecyl and 2-hexyloctyl.

According to one exemplary embodiment, in formula (I), R is chosen from a C12-C16 alkyl radical.

According to one exemplary embodiment, in formula (I), n ranges from 3 to 5.

Tetraethoxylated lauryl acrylate may be chosen as monomer of formula (I).

The monomer (3) of formula (I) may be present in the anionic terpolymer in molar proportions ranging from 0.1 mol % to 10 mol %, for example from 0.5 mol % to 5 mol %.

According to one exemplary embodiment, the anionic terpolymer is crosslinked and/or branched with a diethylenic or polyethylenic compound in the proportion expressed relative to the total amount of monomers used, ranging from 0.005 mol % to 1 mol %, such as from 0.01 mol % to 0.5 mol %, or from 0.01 mol % to 0.25 mol %.

The crosslinking agent and/or branching agent may be chosen from ethylene glycol dimethacrylate, diallyloxyacetic acid or a salt thereof, such as sodium diallyloxyacetate, tetraallyloxyethane, ethylene glycol diacrylate, diallylurea, triallylamine, trimethylolpropane triacrylate and methylenebis(acrylamide), or mixtures thereof.

The anionic terpolymer may contain additives such as complexing agents, transfer agents, or chain-limiting agents.

In various exemplary embodiments, anionic terpolymers of 2-methyl-2-[(1-oxo-2-propenyl)amino]-1-propanesulfonic acid partially or totally salified in the form of the ammonium salt, N,N-dimethylacrylamide and tetraethoxylated lauryl acrylate crosslinked with trimethylolpropane triacrylate, of INCI name Polyacrylate Crosspolymer-6, such as the product sold under the trade name Sepimax Zen® by the company SEPPIC, may be used.

The anionic terpolymer may be present in the booster composition in an amount sufficient to thicken and stabilize the booster composition. By way of example, the anionic terpolymer may be present in the booster composition in an amount up to 10%, such as up to 9%, up to 8%, up to 7%, up to 6%, up to 5%, up to 4%, up to 3%, up to 2%, up to 1%, up to 0.5%, or up to 0.1%, by weight of the booster composition. It is to be understood that the anionic terpolymer can be present in the booster composition in a range having any of the aforementioned numbers as either a low or high end of the range.

For example, the anionic terpolymer may be present in the booster composition in an amount ranging from 0.1% to 10% by weight of the booster composition. In further exemplary embodiments, the anionic terpolymer is present in an amount ranging from 0.5-10%, from 0.5-8%, from 0.5-5%, from 0.5-3%, from 1-10%, from 1-8%, from 1-5%, or from 1-3%, including ranges and sub-ranges therebetween, by weight of the booster composition.

The booster composition may further comprise a cosmetically acceptable aqueous medium in an amount up to 99%, by weight of the booster composition. For example, the booster composition may comprise water in an amount up to 99% by weight of the booster composition, such as up to 98%, up to 97%, up to 96%, up to 95%, up to 94%, up to 93%, up to 92%, up to 91%, up to 90%, up to 89%, up to 88%, up to 87%, up to 86%, up to 85%, up to 84%, up to 83%, up to 82%, up to 81%, up to 80%, up to 79%, up to 78%, up to 77%, up to 76%, up to 75%, up to 74%, up to 73%, up to 72%, up to 71%, up to 70%, up to 65%, or up to 60%, by weight of the booster composition. It is to be understood that the water can be present in the booster composition in a range having any of the aforementioned numbers as either a low or high end of the range. For example, the booster composition may comprise water in an amount ranging from 70% to 99%, by weight of the booster composition.

The booster composition may optionally comprise additional components, as long the additional, optional components do not, or do not significantly, negatively affect the stability or other desired properties (e.g. effervescent effect) of the booster composition.

As non-limiting examples, the booster composition may comprise pigments, additional thickeners, film formers, antioxidants, essential oils, botanical extracts, fragrances, preserving agents, emollients, moisturizers, and vitamins.

By way of example, pigments that can be used in the booster composition include pearlescent pigments mica coated with titanium oxide, mica coated with titanium dioxide, bismuth oxychloride, titanium oxychloride, colored pearlescent pigments such as titanium mica with iron oxides, titanium mica with ferric blue, chromium oxide and the like, titanium mica with an organic pigment of the above-mentioned type as well as those based on bismuth oxychloride and mixtures thereof. In one exemplary embodiment, mica and/or titanium dioxide are chosen.

If present, the additional, optional components may be present, individually or in total, in amounts up to 5%, such as up to 4%, up to 3%, up to 2%, up to 1%, up to 0.5%, up to 0.05%, or up to 0.01%, by weight of the booster composition. It is to be understood that the additional, optional components may individually or in total be present in the booster composition in a range having any of the aforementioned numbers as either a low or high end of the range. For example, the booster composition may comprise additional, optional components individually or in total in an amount ranging from 0.01% to 5%, such as 0.05% to 2%, or 0.1% to 1%, by weight of the booster composition.

The booster composition may be in any form. For example, the booster composition may be a solution, an emulsion, a gel, a powder, a free-flowing powder, granules, tablets, and the like. In one exemplary embodiment, the booster composition is in dry form, e.g. a powder or granules, which can be mixed with a solvent such as water at or near the time of use. In yet a further exemplary embodiment, the booster composition is an aqueous solution.

In one exemplary and non-limiting embodiment, the booster composition is a stable aqueous composition comprising sodium bicarbonate in an amount ranging from about 5% to about 20%, Polyacrylate Crosspolymer-6 in an amount ranging from about 1% to about 3%, and mica and titanium dioxide in a total combined amount ranging from about 0.05% to about 1%.

According to various embodiments, the booster composition may have a pH of 10.5 or lower, such as 10.0 or lower, 9.5 or lower, 9.0 or lower, 8.5 or lower, or 8.0 or lower. For example, the booster composition may have a pH ranging from about 7.5 to about 10.5, such as from about 8.0 to about 10.0, from about 8.3 to about 9.5, or from about 8.5 to about 9.0.

### Shampoo Composition

The shampoo composition may be any conventional shampoo composition comprising cleansing surfactants, and optionally further comprising conditioning agents. The shampoo composition may, for example, be a conventional liquid shampoo composition.

The shampoo composition may also be those that employ anionic (cleansing) surfactants chosen from sulfate-based compounds, non-sulfate-based compounds, and mixtures thereof. Thus, shampoo compositions may be sulfate-based shampoos or sulfate-free shampoos.

Useful and non-limiting cleansing surfactants may be chosen from anionic, amphoteric, and nonionic cleansing surfactants. For example, useful anionic cleansing surfactants may be chosen from salts of each of alkyl sulfates, alkyl ether sulfates, acyl isethionates, acyl glycinates, acyl taurates, acyl amino acids, acyl sarcosinates, sulfosuccinates, sulfonates, isethionates, wherein the alkyl and acyl groups of these compounds comprise from 6 to 40 carbon atoms. Exemplary anionic cleansing surfactants include Sodium Laureth Sulfate, Sodium Lauryl Sulfate, Ammonium Lauryl Sulfate, Ammonium Laureth Sulfate, Sodium Cocoyl Isethionate, Sodium Lauroyl Methyl Isethionate, Sodium Cocoyl Methyl Isethionate, Tea-Cocoyl Glutamate, Sodium Lauroyl Glutamate, Disodium Cocoyl Glutamate, Sodium Cocoyl Glutamate, Sodium Cocoyl Alaninate, Sodium Cocyl Glycinate, Disodium Laureth Sulfosuccinate, Sodium Lauryl Sulfoacetate (and) Disodium Laureth Sulfosuccinate, Lauryl Phosphate, Lauryl Phospate and Potassium Phosphate, Sodium Methyl Cocoyl Taurate, Sodium Cocoyl Amino Acids, Sodium Cocoyl Apple Amino Acids, Sodium Lauroyl Sarcosinate, Sodium Cocoyl Sarcosinate, Lauryl Glucoside, Sodium Lauryl Glucose Carboxylate, and mixtures thereof.

Further useful and non-limiting cleansing surfactants may be chosen from amphoteric surfactants, such as betaines, sultaines, amphoacetates, amphoproprionates, and mixtures thereof. Exemplary amphoteric surfactants include Coco Betaine, Cocamidopropyl Betaine, Cocamidopropyl betaine and glyceryl laurate capryl/capramidopropyl betaine, Lauryl betaine, Cocamidopropyl hydroxysultaine, sodium cocoamphodiacetate, sodium cocoamphoacetate, sodium cocoamphopropionate, and mixtures thereof.

Further useful and non-limiting cleansing surfactants may be chosen from nonionic surfactants, such as alkyl polyglucosides, glycol ethers, amine oxides, and mixtures thereof. Exemplary nonionic cleansing surfactants include lauryl glucoside, decyl glucoside, coco glucoside, Cocamide MIPA, PEG-8 glyceryl laurate, polysorbate-40, polyglyceryl-5 laurate, lauryl amine oxide, and mixtures thereof.

Useful and non-limiting conditioning agents include cationic surfactants and polymers. For example, polyquaterium-10, cetrimonium chloride, behentrimonium chloride, behentrimonium methosulfate, steartrimonium chloride, stearalkonium chloride and dicetyldimonium chloride, cetrimonium chloride, quaternium-22, behenylamidopropyl-2, 3-di-hydroxypropyldimethylammonium chloride, palmitylamidopropyltrimethylammonium chloride, and chloride and methyl sulfate of diacyloxyethyl-dimethylammonium, of diacyloxyethyl-hydroxyethyl-methylammonium, of monoacyloxyethyl-dihydroxyethyl-methylammonium, of triacyloxyethyl-methylammonium, of monoacyloxyethyl-hydroxyethyl-dimethyl-ammonium, distearyldimethylammonium chloride, oleocetyldimethylhydroxyethylammonium chloride, stearamidopropyldimethyl (myristyl acetate) ammonium chloride, di(C1-C2 alkyl) (C12-C22 alkyl)hydroxy(C1-C2alkyl)ammonium salt, or alkyltrimethylammonium salt in which the alkyl radical comprises 12 to 24 carbon atoms, propanetallowdiammonium dichloride, behentrimonium methosulfate, quaternium-83, quaternium-87, and mixtures thereof may be chosen. In various embodiments, polyquaternium-10, acacia Senegal gum, and/or guar hydroxypropyltrimonium chloride may be chosen.

In various embodiments, it may be beneficial to choose a low-pH shampoo, such as a shampoo having a pH of 5.5 or lower, such as 5.0 or lower, 4.5 or lower, 4.0 or lower, 3.5 or lower, or 3.0 or lower. For example, the low-pH shampoo may have a pH ranging from 3.0 to 3.5, such as 3.1 to 3.4, 3.1 to 3.3, or about 3.2 to 3.3. Optionally, the shampoo composition may contain at least one acid.

In various exemplary and non-limiting embodiments, the at least one acid may be chosen from at least one non-polymeric and non-thiol, mono-, di-, and/or tricarboxylic acids, and/or salts thereof, or a mixture thereof. In one embodiment, the non-polymeric mono, di, and tricarboxylic acids have a molecular weight of less than about 500 g/mol, such as less than about 400 g/mol, or less than about 300 g/mol.

In various embodiments, the at least one acid may be chosen from monocarboxylic acids. For example, the shampoo composition may include at least one monocarboxylic acid selected from lactic acid, formic acid, acetic acid, propionic acid, butyric acid, valeric acid, caproic acid, entanthic acid, caprylic acid, pelargonic acid, capric acid, undecylic acid, lauric acid, tridecylic acid, lauric acid, tridecylic acid, myristic acid, pentadecylic acid, palmitic acid, margaric acid, stearic acid, nonadecylic acid, arachidic acid, lactic acid, a salt thereof, and a mixture thereof. In some embodiments, lactic acid and/or a salt thereof is a preferred monocarboxylic acid.

In various embodiments, the at least one acid may be chosen from dicarboxylic acids. For example, the shampoo composition may include at least one dicarboxylic acid selected from oxalic acid, malonic acid, malic acid, glutaric acid, citraconic acid, succinic acid, adipic acid, tartaric acid, fumaric acid, maleic acid, sebacic acid, azelaic acid, dodecanedioic acid, phthalic acid, isophthalic acid, terephthalic acid, 2,6-naphthalene dicarboxylic acid, a salt thereof, and a mixture thereof. In some embodiments, malic acid, glutaric acid, a salt thereof, or a mixture thereof are preferred dicarboxylic acids.

In various embodiments, the at least one acid may be chosen from tricarboxylic acids. For example, the shampoo composition may include at least one tricarboxylic acid selected from citric acid, isocitric acid, aconitric acid, propane-1,2,3-tricarboxylic acid, benzene-1,3,5-tricarboxylic acid, a salt thereof, and a mixture thereof. In some embodiments, citric acid and/or a salt thereof are preferred tricarboxylic acids.

In one embodiment, the shampoo compositions include two or more non-polymeric and non-thiol, mono-, di-, and/or tri-carboxylic acids, and/or salts thereof. The two or more non-polymeric and non-thiol, mono-, di-, and/or tric-arboxylic acids may be, for example, any two or more of those listed above. In one embodiment, the shampoo compositions include at least two monocarboxylic acids and/or salts thereof; at least two dicarboxylic acids and/or salts thereof; or at least two tricarboxylic acids and/or salts thereof.

In further embodiments, the shampoo compositions include at least one monocarboxylic acid and/or salts thereof and at least one dicarboxylic acid and/or salts thereof; at least one monocarboxylic acid and/or salts thereof and at least one tricarboxylic acid and/or salts thereof; at least one dicarboxylic acid and/or salts thereof and at least one tricarboxylic acid and/or salts thereof; or at least one monocarboxylic acid and/or salts thereof and at least one dicarboxylic acid and/or salts thereof and at least one tricarboxylic acid and/or salts thereof.

When present, the at least one acid may be in the shampoo composition in an individual or total combined amount ranging up to 5%, such as up to 4%, up to 3%, up to 2%, up to 1%, up to 0.5%, or up to 0.01%, by weight of the shampoo composition. It is to be understood that the acid may be present in the shampoo composition in a range having any of the aforementioned numbers as either a low or high end of the range. For example, the shampoo composition may comprise at least one acid in an individual or total combined amount ranging from 0.01% to 5%, such as 1% to 4%, or 1% to 3%, by weight of the shampoo composition.

The shampoo composition may optionally comprise other additional components traditionally used in shampoo compositions, provided they don't, or don't significantly, interfere with the effervescent effect of the booster composition when used in conjunction with the shampoo. For example, the shampoo composition may optionally additionally comprise natural and synthetic oils, humectants, shine agents, fillers, colorants, pigments, chelating agents, sequestering agents, fragrances, preservatives, stabilizers, thickeners, viscosity adjusters, conditioning agents, pH adjusters, salts, and mixtures thereof.

By way of example only, liquid shampoo compositions such as those disclosed in, for example, U.S. Publication Nos. 2018/0055751, 2017/0281522, and 2017/0151157, and in WO2012/072765 may be used.

### Effervescent Cleansing Composition and Methods of Cleansing Keratin Fibers

The system comprised of a first booster composition and a second shampoo composition may be mixed to form an effervescent cleansing composition that can be used to cleanse keratin fibers, such as hair. According to various embodiments, the booster composition and shampoo composition may be mixed at or near the time of use to substantially immediately form a foaming composition, which can be applied to the keratin fibers to cleanse the keratin fibers. In various embodiments, the hair may be subsequently optionally rinsed.

As used herein, mixing “at or near the time of use” is intended to convey that the mixing typically occurs substantially immediately before use, for example up to 1 minute before use, such as up to 2 minutes, up to 5 minutes, up to 10 minutes, up to 15 minutes, up to 20 minutes, up to 30 minutes, up to 60 minutes, up to 2 hours, up to 6 hours, up to 12 hours, or up to 24 hours before use. Although not required, the mixing at or near the time of use may be performed or effected by the user, who may be an individual cleansing their own hair or the hair of another person (e.g. at a salon). Any form of mixing of the booster composition and shampoo composition is contemplated.

As used herein, language indicating that the booster composition is “used in conjunction with” a shampoo composition is intended to mean that the booster composition and shampoo composition are applied to the hair at substantially the same time, preferably without an intermediate step of rinsing. In one exemplary embodiment, the booster composition and liquid shampoo composition may be dispensed from separate containers and mixed in the user's hand or on the user's head. In another exemplary embodiment, the booster composition and liquid shampoo composition may be dispensed substantially simultaneously from a multi-chamber container to the user's hand or head. Thus, “mixed” as used herein should not be limited to mixing prior to application to the keratin fibers. It is to be understood, therefore, that the disclosure is not intended to be limiting as to the manner or order of dispensing or mixing of the booster and/or shampoo compositions.

According to various embodiments, the ratio of booster composition to shampoo composition mixed to form the effervescent cleansing composition can range from about 1:20 to 1:2. For example, the ratio of booster composition:shampoo composition can be about 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, or 1:2, including all ranges and subranges thereof. It is to be understood that the ratio of booster composition to shampoo composition mixed to form the effervescent cleansing composition can be a range having any of the aforementioned numbers as either a low or high end of the range.

According to various embodiments, the effervescent cleansing composition may have a pH of 7.0 or lower, such as 6.5 or lower, 5.5 or lower, 5.0 or lower, 4.5 or lower, or 4.0 or lower. For example, the effervescent cleansing composition may have a pH ranging from 4.0 to 7.0, such as about 4.5, about 5.0, about 5.5, about 6.0, about 6.5, or about 7.0.

It should be understood that the booster composition, shampoo composition, and effervescent cleansing composition according to the disclosure are not intended to be hair styling compositions, and the methods according to the disclosure are not intended to be hair styling methods.

### Kits and Containers

The disclosure also relates to kits and containers comprising a booster composition, and optionally a shampoo composition, according to the disclosure.

According to various embodiments, kits for cleansing the hair may be multi-compartment kits, where the compartments are mutually separate. For example, the kits may comprise at least two compartments, with a first compartment containing a booster composition and a second compartment containing a shampoo composition according to the disclosure.

The compartments of kits according to the disclosure can be in any configuration, without limitation. For example, they can be a bottle, a tube, a sachet, an ampoule, or any other container configured to contain the booster and shampoo compositions mutually separately in the kit.

One exemplary embodiment of a kit for cleansing the hair comprises:

a first compartment containing a booster composition comprising:

i. at least one carbon dioxide-generating compound; and

ii. at least one anionic terpolymer;

and a second compartment containing a shampoo composition.

A further exemplary embodiment of a kit for cleansing the hair comprises:

a first compartment containing a booster composition comprising an aqueous medium comprising:

i. sodium bicarbonate; and

ii. polyacrylate crosspolymer-6;

and a second compartment containing a shampoo composition comprising at least one cleansing surfactant and at least one acid.

Various embodiments of containers for containing both the booster composition and shampoo composition according to the disclosure can be used. For example, a multi-chamber container may be chosen, where a first chamber comprises a booster composition according to the disclosure and a second chamber comprises a shampoo composition.

By way of example only, a container having at least two mutually separate storage chambers, the first of which contains a booster composition and the second of which contains a shampoo composition, each of whose contents can be removed simultaneously from their respective chambers by means of two separate outlets. In such an embodiment, the booster and shampoo compositions may, for example, be dispensed individually but simultaneously into a user's hand or onto a user's head, and subsequently mixed. In various embodiments, the ratio of the discharged amount of booster composition to shampoo composition may be predetermined to be delivered simultaneously to the user and then mixed in the user's hand or on the user's head.

As a further example, a container having at least two mutually separate storage chambers and a third, separate mixing chamber or common outlet, the first storage chamber containing a booster composition and the second storage chamber containing a shampoo composition, each of whose contents can be removed simultaneously to a third mixing chamber or common outlet, whereby mixing optionally occurs in the third mixing chamber or common outlet before being dispensed to the user via a single outlet. In various embodiments, the ratio of the discharged amount of booster composition to shampoo composition is predetermined to be delivered, for example in an already-mixed form, to the user. Such multi-chamber containers may be, for example, pump foamer containers or squeeze foamer containers.

One exemplary embodiment of a multi-chamber container for cleansing the hair comprises: a first chamber containing a booster composition comprising:

i. at least one carbon dioxide-generating compound; and

ii. at least one anionic terpolymer;

and a second chamber containing a shampoo composition.

A further exemplary embodiment of a multi-chamber container for cleansing the hair comprises: a first chamber containing a booster composition comprising an aqueous medium comprising:

i. sodium bicarbonate; and

ii. polyacrylate crosspolymer-6;

and a second chamber containing a liquid shampoo composition comprising an acid.

In yet further exemplary embodiments, the container may be a bottle, a tube, a sachet, an ampoule, or any other container configured to contain a booster composition, separate from any container comprising a shampoo composition. In such embodiments, the booster composition can be used in conjunction with any shampoo composition of the user's choice.

According to various exemplary embodiments, advantages associated with the effervescent cleansing compositions include excellent hair cleansing and scalp care, added fullness and volume to the hair, and quick foaming properties. It should be understood, however, that embodiments that do not impart one or more of the advantages are still within the scope of the disclosure.

It is to be understood that all definitions herein are provided for the present disclosure only.

It to be understood that, as used herein the terms “the,” “a,” or “an,” mean “at least one,” and should not be limited to “only one” unless explicitly indicated to the contrary. Thus, for example, reference to “the acid” includes embodiments having two or more acids unless the context clearly indicates otherwise.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that any particular order be inferred.

While various features, elements or steps of particular embodiments may be disclosed using the transitional phrase “comprising,” it is to be understood that alternative embodiments, including those that may be described using the transitional phrases “consisting” or “consisting essentially of,” are implied. Thus, for example, implied alternative embodiments to a method that comprises A+B+C include embodiments where a method consists of A+B+C and embodiments where a method consists essentially of A+B+C. As described, the phrase “at least one of A, B, and C” is intended to include “at least one A or at least one B or at least one C,” and is also intended to include “at least one A and at least one B and at least one C.”

All ranges and amounts given herein are intended to include sub-ranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. The term “about” is used herein to indicate a difference of up to +/−10% from the stated number, such as +/−10%, +/−9%, +/−8%, +/−7%, +/−6%, +/−5%, +/−4%, +/−3%, +/−2%, or +/−1%.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements. The examples that follow serve to illustrate embodiments of the present disclosure without, however, being limiting in nature.

It will be apparent to those skilled in the art that various modifications and variations can be made in the booster composition, shampoo composition, effervescent cleansing composition, and methods, kits, and containers of the invention without departing from the spirit or scope of the invention. Thus, it is intended that the present invention covers the modifications and variations of this invention provided that they come within the scope of the claims and their equivalents.

### EXAMPLES

The ingredient amounts in the composition/formulations described below are expressed in % by weight, based on the total weight of the specified composition, unless otherwise indicated.

### Example 1: Booster Compositions

Table 1 below provides examples of booster compositions.

TABLE 1

| | Booster | Booster | Booster | Booster |
|---|---|---|---|---|
| | 1A | 1B | 1C | 1D |
| Sodium | 19 | 5 | 5 | 5 |
| Bicarbonate | | | | |
| Pearl Pigment: | 0.2 | — | — | — |
| Mica (and) | | | | |
| Titanium dioxide | | | | |
| Polyacrylate | 1.5 | 2 | 1.8 | 1.5 |
| crosspolymer-6 | | | | |
| Water | q.s. 100 | q.s. 100 | q.s. 100 | q.s. 100 |

Each booster composition was prepared by hydrating polyacrylate crosspolymer-6 in water until it was completely dispersed. Sodium bicarbonate and pearl pigments (mica and titanium dioxide) were then added and mixed in.

### Example 2: Shampoo Compositions

Table 2 provides examples of shampoo compositions useful according to the disclosure.

TABLE 2

| | Shampoo | Shampoo | Shampoo | Shampoo |
|---|---|---|---|---|
| | 2A | 2B | 2C | 2D |
| Citric Acid | — | — | 2 | 2 |
| Salicylic Acid | 0.2 | 0.2 | 0.2 | 0.2 |
| Sodium Chloride | 1 | 1 | 1.7 | 1.7 |
| Anionic | 15 | 15 | 8.4 | 8.4 |
| Surfactants, | | | | |
| Sodium laureth | | | | |
| sulfate, Sodium | | | | |
| lauryl sulfate | | | | |
| Amphoteric | — | — | 2.4 | 2.4 |
| Surfactant, | | | | |
| Cocoamidopropyl | | | | |
| Betaine | | | | |
| Nonionic | 3 | 3 | — | — |
| surfactant, | | | | |
| Cocamide MIPA | | | | |
| Fragrance | 0.8 | 0.8 | — | 1 |
| Conditioning | 0.3 | 0.4 | 0.6 | 0.6 |
| agents/thickening | | | | |
| agents | | | | |
| Preservatives | 0.5 | 0.5 | 0.5 | 0.5 |
| Non-water | 3 | 3 | 0.5 | 0.5 |
| solvent(s) | | | | |
| Water | q.s. 100 | q.s. 100 | q.s. 100 | q.s. 100 |

Each of the shampoo compositions was prepared by completely hydrating a conditioning agent in water. Then sodium laureth sulfate and cocoamidopropyl betaine were added and mixed in well. After mixing in the surfactants, a preservative and the acids were added to the mixture. The pH was adjusted to 3.15-3.3, and viscosity of the mixture was adjusted as necessary.

### Example 3: Effervescent Cleansing Compositions

The effervescent cleansing compositions in Table 3 were prepared.

TABLE 3

| | Cleansing | Cleansing | Cleansing | Cleansing |
|---|---|---|---|---|
| | Compo- | Compo- | Compo- | Compo- |
| | sition | sition | sition | sition |
| INGREDIENT | 3A | 3B | 3C | 3D |
| Sodium | 2.8 | 2.8 | 2.8 | 2.8 |
| Bicarbonate | | | | |
| Sodium | 0.9 | 0.9 | 1.0 | 1.0 |
| Chloride | | | | |
| Citric Acid | — | — | 1.71 | 1.71 |
| Salicylic Acid | 0.2 | 0.2 | 0.2 | 0.2 |
| Pearl pigments | 0.025-0.03 | 0.025-0.03 | 0.025-0.03 | 0.025-0.03 |
| (Mica and | | | | |
| Titanium | | | | |
| dioxide) | | | | |
| Polyacrylate | 0.22 | 0.22 | 0.22 | 0.22 |
| crosspolymer-6 | | | | |
| Conditioning | 0.26 | 0.34 | 0.47 | 0.47 |
| Agents/ | | | | |
| Thickening | | | | |
| Agents | | | | |
| Anionic | 12.8 | 12.8 | 7.2 | 7.2 |
| Surfactants, | | | | |
| Sodium laureth | | | | |
| sulfate, | | | | |
| Sodium lauryl | | | | |
| sulfate | | | | |
| Amphoteric | | | 2.1 | 2.1 |
| Surfactant, | | | | |
| Cocoamidopropyl | | | | |
| Betaine | | | | |
| Nonionic | 2.6 | 2.6 | | |
| surfactant, | | | | |
| Cocamide Mipa | | | | |
| Fragrance | 0.7 | 0.7 | — | 0.8 |
| Preservative | 0.6 | 0.6 | 0.6 | 0.6 |
| Water | q.s. 100 | q.s. 100 | q.s. 100 | q.s. 100 |

The Cleansing Compositions 3A, 3B, 3C, and 3D were prepared by combining the Booster 1A with Shampoos 2A, 2B, 2C, and 2D, respectively. Each of Shampoos 2A, 2B, 2C, and 2D was independently dispensed from its container into the palm of a user's hand in approximately a two-quarter-size amount (about 20 g). Booster 1A was then dispensed from its container in an amount of a full pump, which was predetermined at about 3.5 g. Booster 1A was manually mixed with each shampoo composition in the palm of the user's hand until the product transformed into an effervescent, foaming composition. The Shampoo and the Booster were easy to mix together and created abundant rich lathers. The resultant mixtures provided effervescent Cleansing Compositions A-D, each of which had a pleasant, mousse-like texture with shimmery and luminescent effects.

### Example 4: Application to Hair

Cleansing Compositions A-D were applied to the hair, immediately producing abundant rich suds in the hair. After lathering, the composition was rinsed out of the hair and scalp using water, and the hair was allowed to dry.

The users reported that the effervescent composition felt luxurious and unique, had a pleasant sensoriality, and was easy to rinse from the hair. Users also reported that the hair after use looked and felt clean, appeared fuller and more airy without the need for additional products typically used to achieve volume, but not dry or brittle. The hair was soft to the touch with no hard or crusty residue. The hair was sleek, smooth, and silky on all textures of hair.

1. A system comprising:
(a) a booster composition comprising:
i. at least one carbon dioxide-generating compound, and
ii. at least one anionic terpolymer; and
(b) a shampoo composition;
wherein the booster composition and the shampoo composition are combined to form an effervescent cleansing composition at or near the time of use.

2. The system of claim 1, wherein the at least one carbon dioxide-generating compound is chosen from carbonate or bicarbonate salts of alkaline metals or alkaline earth metals.

3. The system of claim 2, wherein the carbonate or bicarbonate salts of alkaline metals or alkaline earth metals are chosen from sodium carbonate, potassium carbonate, magnesium carbonate, calcium carbonate, sodium bicarbonate, potassium bicarbonate, magnesium bicarbonate, calcium bicarbonate, and mixtures thereof.

4. The system of claim 1, wherein the anionic terpolymer is chosen from linear or branched and/or crosslinked terpolymers comprising at least one monomer (1) bearing an acid function in free form, partially or totally salified with a nonionic monomer (2) chosen from N,N-dimethylacrylamide and 2-hydroxyethyl acrylate, and at least one polyoxyethylenated alkyl acrylate monomer (3) of formula (I): 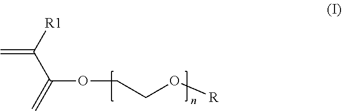
wherein:
R1 represents a hydrogen atom,
R represents a linear or branched C2-C5 alkyl radical, and
n represents a number ranging from 1 to 10.

5. The system of claim 1, wherein the booster composition comprises from about 5-25% by weight of carbon dioxide-generating compound, relative to the total weight of the booster composition.

6. The system of claim 1, wherein the booster composition is in the form of a solution, an emulsion, a gel, a powder, a free-flowing powder, granules, or tablets.

7. The system of claim 1, wherein the shampoo composition comprises at least one acid.

8. The system of claim 7, wherein the at least one acid is chosen from monocarboxylic acids and/or salts thereof, dicarboxylic acids and/or salts thereof, tricarboxylic acids and/or salts thereof, and mixtures thereof.

9. The system of claim 1, wherein the shampoo composition comprises at least one surfactant chosen from anionic, non-ionic, amphoteric surfactants, and mixtures thereof.

10. The system of claim 1 comprising:
(a) a booster composition comprising, in an aqueous medium:
i. at least one carbon dioxide-generating compound chosen from sodium carbonate, potassium carbonate, magnesium carbonate, calcium carbonate, sodium bicarbonate, potassium bicarbonate, magnesium bicarbonate, calcium bicarbonate, and mixtures thereof,
ii. at least one anionic terpolymer chosen from linear or branched and/or crosslinked terpolymers comprising at least one monomer (1) bearing an acid function in free form, partially or totally salified with a nonionic monomer (2) chosen from N,N-dimethylacrylamide and 2-hydroxyethyl acrylate, and at least one polyoxyethylenated alkyl acrylate monomer (3) of formula (I): 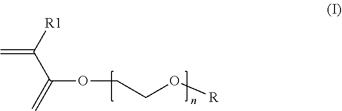
wherein:
R1 represents a hydrogen atom,
R represents a linear or branched C2-C5 alkyl radical, and
n represents a number ranging from 1 to 10; and
iii. at least one pigment; and
(b) a shampoo composition comprising:
i. at least one cleansing surfactant chosen from anionic, non-ionic, amphoteric surfactants, and mixtures thereof;
ii. optionally, at least one conditioning agent; and
iii. at least one acid chosen from chosen from monocarboxylic acids and/or salts thereof, dicarboxylic acids and/or salts thereof, tricarboxylic acids and/or salts thereof, and mixtures thereof;
wherein the booster composition and the shampoo composition are combined to form an effervescent cleansing composition at or near the time of use.

11. A method for cleansing keratin fibers comprising:
(1) mixing a booster composition comprising at least one carbon dioxide-generating compound and at least one anionic terpolymer with a shampoo composition to form an effervescent cleansing composition;
(2) applying the effervescent cleansing composition to the keratin fibers; and
(3) optionally rinsing the effervescent cleansing composition from the keratin fibers.

12. The method of claim 11, wherein the at least one carbon dioxide-generating compound in the booster composition is chosen from carbonate or bicarbonate salts of alkaline metals or alkaline earth metals.

13. The method of claim 12, wherein the carbonate or bicarbonate salts of alkaline metals or alkaline earth metals are chosen from sodium carbonate, potassium carbonate, magnesium carbonate, calcium carbonate, sodium bicarbonate, potassium bicarbonate, magnesium bicarbonate, calcium bicarbonate, and mixtures thereof.

14. The method of claim 11, wherein the anionic terpolymer is chosen from linear or branched and/or crosslinked terpolymers comprising at least one monomer (1) bearing an acid function in free form, partially or totally salified with a nonionic monomer (2) chosen from N,N-dimethylacrylamide and 2-hydroxyethyl acrylate, and at least one polyoxyethylenated alkyl acrylate monomer (3) of formula (I): 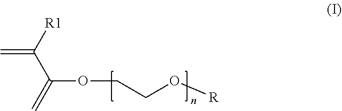
wherein:
R1 represents a hydrogen atom,
R represents a linear or branched C2-C5 alkyl radical, and
n represents a number ranging from 1 to 10.

15. The method of claim 11, wherein the booster composition comprises from about 5-25% by weight of carbon dioxide-generating compound, relative to the total weight of the booster composition.

16. The method of claim 11, wherein the booster composition is in the form of a solution, an emulsion, a gel, a powder, a free-flowing powder, granules, or tablets.

17. The method of claim 11, wherein the shampoo composition comprises at least one acid.

18. The method of claim 17, wherein the at least one acid is chosen from monocarboxylic acids and/or salts thereof, dicarboxylic acids and/or salts thereof, tricarboxylic acids and/or salts thereof, and mixtures thereof.

19. The method of claim 11, wherein the shampoo composition comprises at least one surfactant chosen from anionic, non-ionic, amphoteric surfactants, and mixtures thereof.

20. The method of claim 11 comprising:
(1) mixing:
(a) a booster composition comprising:
i. at least one carbon dioxide-generating compound chosen from sodium carbonate, potassium carbonate, magnesium carbonate, calcium carbonate, sodium bicarbonate, potassium bicarbonate, magnesium bicarbonate, calcium bicarbonate, and mixtures thereof,
ii. at least one anionic terpolymer chosen from linear or branched and/or crosslinked terpolymers comprising at least one monomer (1) bearing an acid function in free form, partially or totally salified with a nonionic monomer (2) chosen from N,N-dimethylacrylamide and 2-hydroxyethyl acrylate, and at least one polyoxyethylenated alkyl acrylate monomer (3) of formula (I): 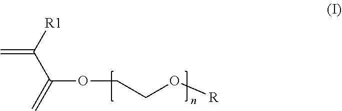
wherein:
R1 represents a hydrogen atom,
R represents a linear or branched C2-C5 alkyl radical, and
n represents a number ranging from 1 to 10, and
iii. at least one pigment, with
(b) a shampoo composition comprising:
i. at least one cleansing surfactant chosen from anionic, non-ionic, amphoteric surfactants, and mixtures thereof;
ii. optionally, at least one conditioning agent; and
iii. at least one acid chosen from chosen from monocarboxylic acids and/or salts thereof, dicarboxylic acids and/or salts thereof, tricarboxylic acids and/or salts thereof, and mixtures thereof;
to form an effervescent cleansing composition;
(2) applying the effervescent cleansing composition to the keratin fibers; and
(3) optionally rinsing the effervescent cleansing composition from the keratin fibers.

21. A system comprising:
(a) a booster composition comprising:
i. sodium bicarbonate, and
ii. polyacrylate crosspolymer-6; and
(b) a shampoo composition comprising at least one cleansing surfactant and at least one acid;
wherein the booster composition and the shampoo composition are combined to form an effervescent cleansing composition at or near the time of use.

22. A kit comprising:
(1) a first compartment comprising a booster composition comprising at least one carbon dioxide-generating compound and at least one anionic terpolymer;
(2) a second compartment comprising a shampoo composition;
wherein the booster composition and the shampoo composition are in mutually separate compartments.

23. A multi-chamber container comprising a first and second separate chambers, wherein:
(1) the first chamber comprises a booster composition comprising at least one carbon dioxide-generating compound and an anionic terpolymer; and
(2) the second chamber comprises a shampoo composition; and
wherein the container is configured so that the booster composition is dispensed from the first chamber and the shampoo composition is dispensed from the second chamber substantially simultaneously to form an effervescent cleansing composition.
